(19) United States
(12) Patent Application Publication
(10) Pub. No.: US 2003/0213169 A1
(43) Pub. Date: Nov. 20, 2003

(54) Method of protecting plants from thrips

(75) Inventor: Wayne Allen, St. Catharines (CA)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Her Majesty in Right of Canada as respresented by the Minister of Agriculture, London (CA)

(21) Appl. No.: 10/145,470

(22) Filed: May 14, 2002

(51) Int. Cl. A01B 79/02; A01C 1/00; A01G 1/00; A01H 3/00; A01B 79/00

(52) U.S. Cl. 47/58.1 R

(57) ABSTRACT

The invention is a method of protecting a plant from thrips comprising the step of applying silica particles to a plant or a plant's surrounding area, wherein the silica particles are fumed silica particles or silica aerogel particles. The act of protecting a plant from thrips includes eradicating thrips, repelling thrips, preventing thrips infestation, inhibiting thrips feeding, inhibiting thrips egg laying, and/or preventing virus acquisition or transmission by thrips.

### FIELD OF THE INVENTION

This invention pertains to a method of protecting plants from thrips.

### BACKGROUND OF THE INVENTION

Thrips (Thripidae, Thysanoptera) are one of the most important and difficult to control pests of greenhouse, field, and orchard crops. They are found throughout the entire United States on numerous types of ornamental and vegetative plants. Most greenhouse, field, vegetable, flower, and orchard crops are infested with at least one of the 6,000 known species of thrips, such as greenhouse thrips, flower thrips, western flower thrips (WFT), onion thrips, soybean thrips, and tobacco thrips. These tiny insects have a reproductive cycle spanning approximately two weeks. Thus, population increases are rapid under favorable conditions.

Thrips cause economic damage and crop losses both directly and indirectly. Thrips feed on all plant parts. Thrips have piercing-sucking mouth parts, and their feeding results in the death of tissues or deformation of flowers, leaves, and fruit. Moderate infestations are responsible for slow plant growth and poor yield and fruit quality. Major infestations can result in death of the entire plant.

Moreover, the thrips' rasping leaves a vulnerable spot on the flower or leaf allowing for facile transmission of a virus from the environment to the plant, which can result in further economic damage and crop losses. In addition, thrips can carry plant pathogens in their mouths and directly transfer them from one plant to another as they feed. When one of the several viruses that thrips transmit are present, even minor infestations of thrips can result in significant disease outbreaks and crop losses.

Thrips usually concentrate on rapidly growing tissues such as young leaves, flowers, and terminal buds. The affinity of thrips for such plant parts makes control of the thrips by coverage of a plant with a pesticide difficult.

Conventional chemical pesticides, such as endosulfan, chlorpyrifos, malathion, acephate, bendiocarb, methomyl, and deltamethrin, are effective against eradicating thrips. However, increasing levels of resistance in thrips to these synthetic chemicals is occurring widely. Thrips that are resistant to one chemical pesticide may develop resistance more quickly to a new chemical pesticide. Currently, nine of the twelve chemical pesticides registered for thrips control are unusable or at risk of being unusable due to resistance or because they are dangerous to humans and the environment (e.g., organophosphates, organochlorines, or carbamates).

Alternatives to conventional chemical pesticides are physical pesticides, such as clay, diatomaceous earth products, and silica gels. These alternatives are often more desirable because they are safer to use and are generally less injurious to non-target organisms which may provide some level of biological control of thrips. Further, physical pesticides are active against both pesticide-resistant and pesticide-susceptible thrips, and these products are unlikely to induce their own resistance. However, the effectiveness of these products is usually limited because they are difficult or impossible to apply evenly and thoroughly as a dust over a crop due to their high bulk density and a strong tendency to clump. Diatomaceous earth works essentially by abrading away the waxy coating on the insect, thereby causing water loss from the insect (see, e.g., U.S. Pat. Nos. 3,159,536; 4,279,895; 4,386,071; and 5,186,935). Other physical pesticides, such as silica gel, are known to kill crawling insects like cockroaches, fleas, termites, mites, and mosquitoes (see, e.g., U.S. Pat. Nos. 3,111,384; 3,124,505; and 3,235,451) by absorbing away the hydrophobic outer layer called the epicuticle. The silica gel adheres well to insects but not to plant leaves.

In view of the foregoing, it would be desirable to provide a safe, yet effective method of protecting plants by controlling thrips. Ideally, the method would take advantage of an inexpensive, cost effective, user- and enviromnentally-safe product that is food-grade with persistent efficacy on the plant and a long storage life. The product should be easily removed from plants and be compatible with other pest management strategies.

The invention provides such a method. These and other advantages of the invention, as well as additional inventive features, will be apparent from the description of the invention provided herein.

### BRIEF SUMMARY OF THE INVENTION

The invention provides a method of protecting a plant from thrips comprising the step of applying silica particles to a plant or its surrounding area, wherein the silica particles are fumed silica particles or silica aerogel particles, to thereby protect the plant from thrips.

### BRIEF DESCRIPTION OF THE DRAWINGS

FIG. 1 is a bar graph of the relative rate of thrips egg laying (% of control) with respect to a 24-hour exposure of the thrips to residues of various chemical pesticides on plant leaf discs.

FIG. 2 is a bar graph representing the number of eggs laid by thrips on leaves of petunia plants left untreated or treated with hydrophilic fumed silica particles.

FIG. 3 is a bar graph of the relative numbers of larvae and adult thrips (% of control) on plant leaves treated with hydrophilic fumed silica particles and various chemical pesticides.

FIG. 4 is a bar graph of the percentage of thrips dropped from plant leaves treated with hydrophilic fumed silica particles and other physical pesticides.

FIG. 5 is a bar graph of the relative counts of larval and adult thrips, as well as viral lesions, (% of control) on plants treated with hydrophilic fumed silica particles and various chemical pesticides.

### DETAILED DESCRIPTION OF THE INVENTION

The invention is a method of protecting a plant from thrips comprising the step of applying silica particles to a plant, especially the leaves, and/or to the area surrounding the plant, wherein the silica particles are fumed silica particles or aerogel particles, to thereby protect the plant from thrips. The act of protecting a plant from thrips includes eradicating thrips, repelling thrips, preventing thrips infestation, inhibiting thrips feeding, inhibiting thrips egg laying, and/or preventing virus acquisition or transmission by thrips.

The silica particles can be any suitable fumed silica particles or silica aerogel particles. The silica particles can be hydrophilic or hydrophilic, but preferably the silica particles are hydrophilic. More preferably the silica particles are hydrophilic fumed silica particles.

The fumed silica particles can be any suitable fumed silica particles. Typically the silica particles will be in the form of particles that are aggregates of smaller, primary particles. Although the primary particles are not porous, the aggregates contain a significant void volume and are capable of rapidly absorbing liquid. These void-containing aggregates enable a coating of such aggregates to retain a significant capacity for liquid absorption even when the aggregate particles are densely packed, which minimizes the inter-particle void volume of the coating.

The silica aerogel particles can be any suitable silica aerogel particles. A “gel” refers to a coherent, rigid, continuous three-dimensional network of colloidal particles. Gels are produced by the aggregation of colloidal particles (typically under acidic conditions when neutralizing salts are absent) to form a three-dimensional gel microstructure. When a gel is dried (i.e., when liquid is removed from the pores) by means in which the coherent gel microstructure is preserved, such as by supercritical drying, a low-density gel or an “aerogel” is formed. A suitable process for the production of an aerogel is described in U.S. Pat. Nos. 2,188,007, 3,122,520, and 3,672,833.

The silica particles can be of any suitable size. Generally, the silica particles (e.g., aggregate or three-dimensional structure) have a mean diameter of at least about 100 nm (e.g., particles having a mean diameter of about 100 nm to 1 μm, more preferably about 100 nm to 500 nm, most preferably about 100 nm to 400 nm, and especially about 200 nm to 300 nm). The silica particles can have any suitable range of individual particle (i.e., aggregate or three-dimensional structure) diameters, such as a relatively broad range or a relatively narrow range. Preferably, all or substantially all of the silica particles have diameters of at least about 30 nm (e.g., all or substantially all of the particles have diameters of about 30 nm to 1 μm). The particles also can be monodispersed. By monodispersed is meant that the individual particles have diameters that are substantially identical. For example, substantially all monodispersed 200 nm particles have diameters in the range of about 190 nm to 210 nm.

It should be noted that the diameter values set forth above for the silica particles refer to the diameters of the aggregates or three-dimensional structures. With respect to the primary particles that make up fumed silica aggregates or three-dimensional structures, it is preferred that the primary particles have a mean diameter of about 100 nm or less (e.g., about 1-100 nm). More preferably, the primary particles have a mean diameter of about 50 nm or less (e.g., about 1-50 nm), even more preferably about 30 nm or less (e.g., about 1-30 nm), and most preferably about 20 nm or less (e.g., about 1-15 nm). In addition, all or substantially all of the primary particles can have diameters smaller than the mean diameter values set forth above. In other words, it is preferred that all or substantially all of the primary particles have diameters of about 100 nm or less (e.g., about 1-100 nm), more preferred that all or substantially all of the primary particles have diameters of about 50 nm or less (e.g., about 1-50 nm), even more preferred that all or substantially all of the primary particles have diameters of about 30 nm or less (e.g., about 1-30 nm), and most preferred that all or substantially all of the primary particles have diameters of about 20 nm or less (e.g., about 1-15 nm).

The silica particles can have any suitable bulk density. Typically, the fumed silica particles have a bulk density that is about 20 kg/m³ to about 115 kg/m³, preferably about 30 kg/m³ to about 50 kg/m³, and more preferably about 35 kg/m³ to about 45 kg/m³. Typically, the silica aerogel particles have a bulk density that is about 0.1 kg/m³ to about 50 kg/m³, preferably about 1 kg/m³ to about 35 kg/m³, and more preferably about 10 kg/m³ to about 20 kg/m³. A relatively low bulk density allows for a facile and thorough application of the fumed silica particles. In comparison, other physical pesticides, such as silica gel and diatomaceous earth, which have relatively high bulk densities, often cake or clump, thereby making application difficult and less effective. Essentially a larger amount of these physical pesticides would have to be applied to obtain the same amount of plant surface area coverage as the fumed silica particles, thereby increasing the cost and labor involved.

The silica particles have a surface area of about 50 m²/g to about 1100 m²/g. The fumed silica particles preferably have a surface area of about 50 m²/g to about 420 m²/g, more preferably about 200 m²/g to about 420 m²/g, even more preferably about 300 m²/g to about 400 m²/g, and most preferably about 300 m²/g to about 350 m²/g. The silica aerogel particles preferably have a surface area of about 200 m²/g to about 1100 m²/g, more preferably about 300 m²/g to about 1000 m²/g, even more preferably about 500 m²/g to about 900 m²/g, and most preferably about 600 m²/g to about 800 m²/g. The surface area described herein is calculated based on the amount of nitrogen adsorbed at five different relative pressures over the range 0.05 to 0.25 atm according to the Brunauer-Emmett-Teller (BET) model, referenced in Gregg, S. J. , and Sing, K. S. W. , “Adsorption, Surface Area and Porosity,” p. 285, Academic Press, New York (1991).

The use of the term “thrips” includes any member of the order Thysanoptera. The order Thysanoptera includes the suborders Terebrantia and Tubulifera, the super families of Aeolothripoidea, Thripoidea, and Merothripoidea, and the families of Aeolothripidae, Heterothripidae, Thripidae, Uzelothripidae, and Phlaeothripidae. Specific varieties of thrips include greenhouse thrips (Heliothrips haemorrhoidalis), banded greenhouse thrips (Hercinothrips femoralis), flower thrips (Frankliniella tritici), Western flower thrips (WFT) (Frankliniella occidentalis), onion or tobacco thrips (Thrips tabaci), citrus thrips (Scirtothrips aurantii and Scirtothrips citri), cereals thrips (Limothrips cerealium), pea thrips (Kakothrips robustus), lily bulb thrips (Liothrips), black hunter thrips (Leptothrips mali), coffee thrips (Diarthrothrips), avocado thrips (Scirtothrips perseae), Thrips palmi, fruit tree thrips (Taeniothrips inconsequens), gladiolus thrips (Taeniothrips simplex), azalea thrips (heterothrips azaleae), olive thrips (Liothrips oleae), six-spotted thrips (Scolothrips sexmaculatus), and cotton thrips (caliothrips sp. and Frankliniella sp.). Members of Thysanoptera are generally characterized by a pretarsus with protusible “bladder”, which balloons out as the leg makes contact with the ground. The foot pad is typically sticky, allowing silica particles to adhere to it and thereby preventing adherence of the foot pad to the plant surface and preventing thrips feeding and egg laying in the plant tissues.

The method of protecting a plant from thrips can be used to protect any plant that is or can be affected by thrips, such as an ornamental plant, tree, food crop, or non-food crop. For example, the plant can be an ornamental plant such as African violet, alstreomeria, aster, azalea, begonia, cacti, calceolaria, celosia, cineraria, cyclamen, chrysanthemum, dalia, exacum, gladiolus, geranium, gerbera, gloxinia, gypsophila, hibiscus, hydrangea, impatiens, kalanchoe, lily, lisianthus, oxalis, primula, petunia, poinsettia, rose, snapdragon, stocks, and stephanotis. The plant can be a crop, either food or non-food, such as citrus, pear, tomato, bean, soybean, cotton, alfalfa, tobacco, onion, cucumber, peanut, cabbage, kale, cauliflower, broccoli, herbs, lettuce, or pepper. The silica particles are effective in controlling thrips on a variety of plant parts, especially plant leaves and including leaf types ranging from shiny, upright leaves, to rough, hairy, drooping leaves.

When the silica particles are applied to plants that are infested with thrips, the thrips generally are eliminated from the plant within 72 hours, preferably within 48 hours, and more preferably within 24 hours. For example, after applying hydrophilic fumed silica particles with a surface area of about 325 m²/g, both adults and larvae were dramatically and significantly (respectively) reduced within 24 hours.

The silica particles can be used both to prevent infestations and to eradicate existing adult and larval thrips. The silica particles can be applied before a plant is exposed to thrips, thereby preventing/limiting an infestation. Alternatively, or in addition, the silica particles can be applied after a plant has been exposed to thrips. This latter approach allows for repulsion of both the current thrips on the plant and newly arriving thrips.

The silica particles can be applied by any suitable technique so as to allow for the placement (e.g., coverage and/or adherence) of the silica particles on the plant and/or the thrips. The silica particles can be applied in dry, powder form using any suitable apparatus, such as a hand-held duster, power duster, or a fixed chamber in which plants are dusted automatically as they are conveyed through the chamber. Preferably the method of protecting a plant from thrips includes having the silica particles applied in a dry form, and more preferably the silica particles are applied in a dry form using a hand-held duster. Alternatively, the silica particles can be applied in a liquid dispersion or suspension using, for example, a hand-held power sprayer or aerosol can, a portable sprayer, or a fixed automated spray-chamber through which plants are conveyed. Preferably the method of protecting a plant from thrips includes having the silica particles applied in a liquid dispersion. The liquid dispersion can comprise about 0.5 g or more, e.g., about 1 g or more, about 2 g or more, about 5 g or more, or about 10 g or more, silica particles per liter of a liquid carrier. The liquid dispersion also can comprise about 40 g or less, e.g., about 30 g or less, about 20 g or less, about 10 g or less, about 5 g or less, or about 1 g or less, silica particles per liter of a liquid carrier. The liquid carrier can be any suitable liquid carrier, particularly a liquid that is inert towards the plant, such as water or water mixed with a suitable dispersant and/or leaf-wetting agent.

In general, the silica particles can be applied to any suitable part of the plant and/or the area surrounding (e.g., adjacent) the plant. For example, the silica particles can be applied to the tip half of some or all of the plant leaves. Desirably, the silica particles are applied to the upper surface and/or the lower surface of some or all of the plant leaves. A preferred method of protecting a plant from thrips includes applying the silica particles to the upper surface of the leaves. When the silica particles are applied as a dry powder, the low bulk density allows for extensive coverage of a plant (e.g., upper surfaces of leaves, lower surfaces of leaves, and stems) with only a light dusting. Since the majority of second instar larvae drop from leaves before developing into pupae and new adults, the silica particles preferably are applied to a plant's surrounding area, such as the dirt or soil around the base of the plant, the plant's container, the floor, ground, and/or bench surfaces (such as within about a 1 meter radius of the plant to be protected, e.g., within about a 0.5 meter radius of the plant to be protected). Applications to the plant or the plant's surrounding area can be the only area of application, or the applications can be to both the plant and the plant's surrounding area. Applications also are preferably made to surfaces and floors in contact with or near the plants, before and especially after the removal of plants from those surfaces and floors or the nearby area, to eradicate thrips that have fallen from plants (e.g., during the removal of infested plants from those surfaces and floors or the nearby area).

When the silica particles are applied to only the tip half of a leaf, typically the efficacy at controlling thrips is substantially the same in comparison to applying the silica particles to the entire upper surface of a leaf. Thrips do not avoid the silica particles, and the specific area of coverage is not as important as with a chemical pesticide. Thrips on a non-treated area of the leaf have been observed freely entering the treated area and become contaminated with the silica particles almost immediately. Once contaminated, the adults and larvae often will attempt to crawl to the lower side of the leaf. Few are able to make the transition, and those that do, adhere only briefly. After a thrips is contaminated with the silica particles, it generally remains as such. In other words, the silica particles adhere to the body of the thrips, in particular the foot pad (tarsal bladder) and wings. Thrips have inflatable foot pads that act as suction cups, and any foreign material that adheres to the foot pads prevents the adherence of the foot pads to the plant surface. Thrips must have good adherence to the plant in order to feed and cannot acquire or transmit viruses if they cannot feed on the plant. In addition, since thrips must puncture a leaf surface with their ovipositor in order to insert eggs below the surface of the plant (in contrast to other insects that lay eggs on the surface of the plant), thrips that do not have good adherence to the plant cannot lay eggs. Accordingly, as a result of the adherence of the silica particles to thrips, the thrips cannot adhere strongly enough to the plant surface to puncture it for purposes of feeding, egg laying, and virus transmission.

The silica particles can be applied to the plant and/or the plant's surrounding area at any suitable rate or in any suitable regimen or protocol. Similarly, any suitable amount of the silica particles can be applied to the plant or the plant's surrounding area to provide protection against thrips. The coverage of the silica particles on the plant should not be so high that light transmission to the plant is significantly reduced, which could impair plant growth and development, such as flower development.

The duration of coverage of the silica particles does not affect efficacy, particularly on bottom-watered plants. The silica particles remain effective as long as they remain relatively dry. While it is preferable to keep the silica particles as dry as possible, silica particles that are partially wet or hydrated are still active in controlling thrips. Aging or weathering the silica particles several weeks to several months typically results in no loss of effectiveness. Once protection is no longer needed, the silica particles easily can be removed from the plant or surrounding area by rinsing with water or any other appropriate liquid, such as a mild soap solution. Since the silica particles are easily washed from the plant, they can be used for short periods or as a temporary measure to reduce thrips populations.

Preferably the method of protecting a plant from thrips includes preventing virus transmission. The silica particles have a high level of efficacy compared to chemical pesticides in controlling virus transmission, in particular transmission by the western flower thrips. Once contaminated with silica particles, a thrips' ability to fly is usually impaired. This effect on flight greatly reduces virus dissemination to other plants. Moreover, even if a contaminated thrips was able to fly to an adjacent plant, it would not be able to adhere strongly enough to the surface of the adjacent plant in order to penetrate the surface of the plant and transmit a virus. This mode of inhibiting virus transmission is applicable to most viruses that can be transmitted by a thrips. In particular, the method of protecting plants from thrips through the application of silica particles prevents (e.g., inhibits) the transmission by thrips of viruses such as tomato spotted wilt and impatiens necrotic spot.

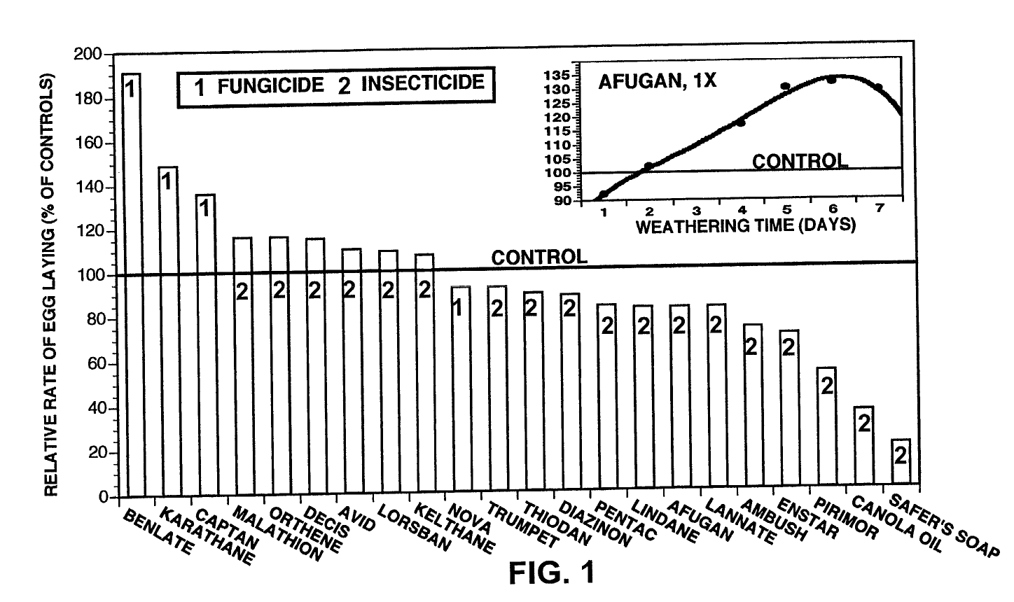

Residues of fungicides and pesticides can have a hormone-like (hormoligosis) effect on adult thrips causing females to lay more eggs and over a shorter time interval compared to non-exposed females. See FIG. 1, which illustrates in bar graph form the effect on thrips egg laying with respect to a 24-hour exposure of the thrips to various chemical residues on plant leaf discs. Some pesticide residues may not induce abnormal egg laying when first applied but will do so over time as the residues weather in greenhouses. The latter phenomenon is exemplified in tests with pyrazophos (see inset graph in FIG. 1), which slightly depressed egg laying over 24 hours but increasingly stimulated egg laying over the ensuing days. Since thrips contaminated with silica particles are inhibited in their ability to adhere to a leaf, the thrips cannot pierce the leaf in order to lay eggs, thereby inhibiting egg laying by the thrips without subsequently stimulating egg laying.

The method of protecting a plant from thrips can include applying the silica particles to the plant, in particular the leaves, and/or the plant's surrounding area in combination with an effective amount of another agent that protects the plant from thrips (i.e., an agent other than the silica particles). This other agent can be any compound effective to eradicate or prevent thrips. The agent includes other physical or chemical pesticides, many of which are known in the art (e.g., insecticidal soap).

Fumed silica particles and silica aerogel particles are believed to cause death to thrips by sorbing away the outer hydrophobic layer called the epicuticle, which is composed of lipid or waxy substances that seal the surface to prevent water loss. Evidence of this mode of action is that adults and larval thrips treated with the silica particles die within several hours if removed from leaves (non-treated thrips can live for 24 hours or longer away from food), but treated thrips survive for several hours if left on the leaf. Presumably, treated thrips on the leaf can replace some of the lost water but not quickly enough to keep up with the rate of dehydration. The thrips slowly lose coordination and mobility, and their bodies noticably shrink as death ensues. The survival time of thrips treated with the silica particles generally decreases as the surrounding air temperature increases.

The surface area of a physical pesticide is believed to play a role in how well it absorbs the waxy cuticle of a thrips. However, the most effective surface area differs among insects, apparently because the composition and physical characteristics of the epicuticular layer differ among insects. For example, cockroaches have a softer coating and thus require a surface area much higher than the silica particles of the invention to absorb the wax. The silica particles used in the invention have a much higher surface area in comparison to other physical pesticides such as diatomaceous earth (which has a surface are of about 30 m²/g). The silica particles have a similar surface area on a weight basis as some silica gels; however, a significant difference resides in the pores. The void space in a silica gel is internal, whereas the void space in the silica particles of the invention is external. While not wishing to be bound by any particular theory, the external void space of the silica particles apparently allows for the more facile migration of the waxy cuticle of the thrips into the silica particles, thereby making it more effective as a pesticide.

The following examples further illustrate the invention but, of course, should not be construed as in any way limiting its scope.

### EXAMPLE 1

This example illustrates the effectiveness of fumed silica particles in controlling egg laying by thrips.

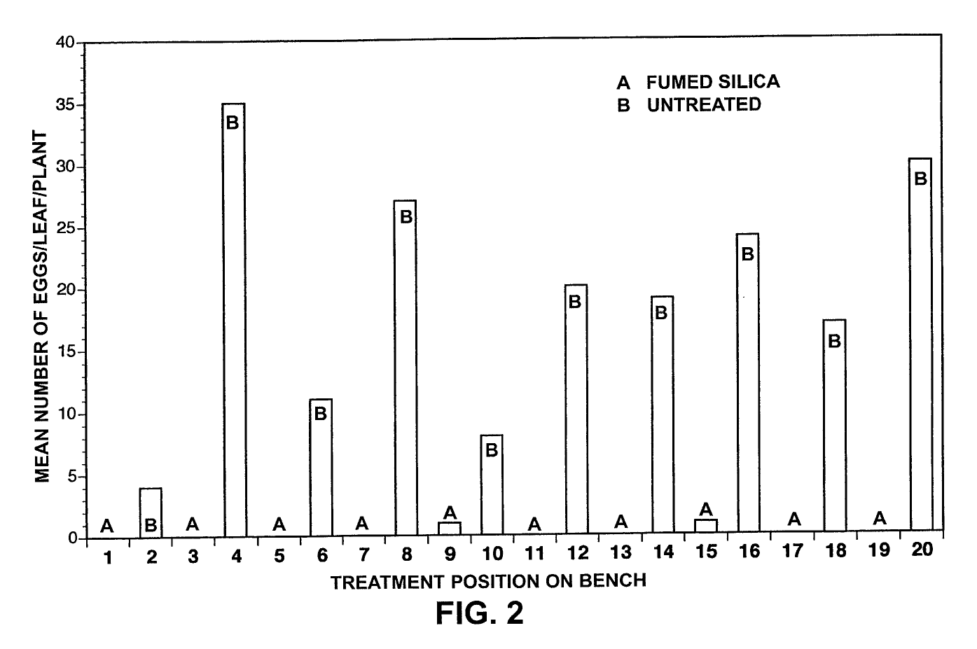

Petunia plants of the same age were trimmed to have a uniform number of leaves before treatment. Half of the plants were dusted with hydrophilic fumed silica particles (about 325 m²/g surface area), and the other half of the plants was left untreated. The treated and untreated plants were alternated in rows in a thrips-infested greenhouse. After seven days, the number of thrips eggs present on each of the plants was counted. The results are plotted in FIG. 2.

The plants treated with the hydrophilic fumed silica particles had 99% fewer thrips eggs than did the untreated plants. These results demonstrate the high level of protection and the uniformity of the effect of hydrophilic fumed silica particles on thrips reproduction under greenhouse conditions.

### EXAMPLE 2

This example illustrates the effectiveness of fumed silica particles in controlling larval thrips in various plants.

Hydrophilic fumed silica particles (about 325 m²/g surface area) were dusted onto the leaves of various plants (“treated plants”). Similar plants were not dusted with the hydrophilic fumed silica particles (“untreated plants”). Treated and untreated plants were alternated in rows on benches in a greenhouse. Thrips-infested potted flowering chrysanthemum plants were placed on each bench in the greenhouse to allow for exposure of the treated and untreated plants to thrips. Larval counts on the treated and untreated plants were taken seven days after the initial exposure of these plants to thrips. The percent reductions of the larval thrips on the treated plants were calculated based on the number of larval thrips on a treated plant compared to an untreated plant. The results are set forth in Table 1.

TABLE 1

| | | % Reduction | Level of |
|---|---|---|---|
| Trial | Plant | in Larvae | Significance (P) |
| 2A | petunia | 92 | 0.0002 |
| 2B | petuma | 93 | <0.0001 |
| 2C | petunia | 96 | <0.0001 |
| 2D | mini roses, “Red Rosa” | 95.4 | 0.0038 |
| 2E | non-flowering chrysanthemum | 99.4 | <0.0001 |
| 2F | pepper | 88.5 | <0.0014 |

These results demonstrate the versatility and effectiveness of fumed silica particles in controlling larval thrips on different plant varieties. The consistency of the results observed for the three trials involving petunia plants demonstrates the reproducibility of the effect.

### EXAMPLE 3

This example illustrates the effectiveness of fumed silica particles in eradicating thrips from infested plants.

Uniformly aged and trimmed petunia plants were placed in rows on benches in a greenhouse containing thrips-infested flowering chrysanthemum plants. After seven days, alternate petunia plants in the rows were dusted with hydrophilic fumed silica particles (about 325 m²/g surface area). Twenty-four or forty-eight hours later, the number of larval thrips on each plant was counted to determine the effect of the silica particles. The percent reductions of the larval thrips on treated plants compared to untreated plants were calculated. The results are set forth in Table 2.

TABLE 2

| | Hours after | % Reduction | Level of |
|---|---|---|---|
| Trial | Application | in Larvae | Significance (P) |
| 3A | 24 | 81 | <0.0001 |
| 3B | 24 | 90 | <0.0001 |
| 3C | 24 | 99.7 | <0.0001 |
| 3D | 48 | 77 | <0.0001 |

These results demonstrate the effectiveness of fumed silica particles in eradicating thrips from infested plants.

### EXAMPLE 4

This example illustrates the effectiveness of eradicating thrips on a plant with fumed silica particles in comparison to chemical pesticides.

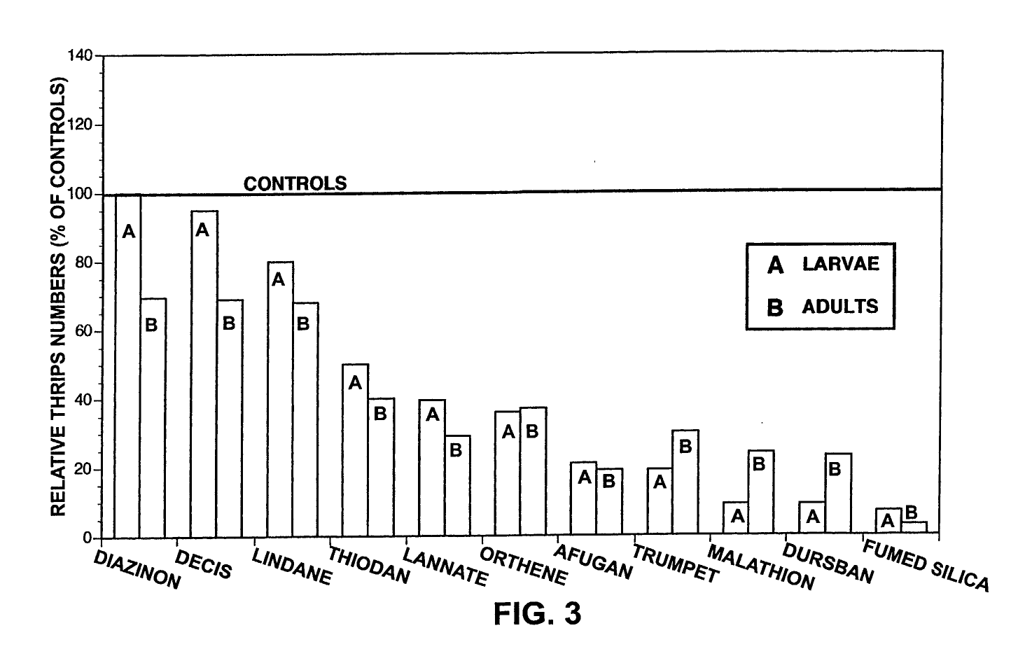

Uniformly aged and trimmed petunia plants were exposed to thrips in a greenhouse containing thrips-infested flowering chrysanthemum plants. After exposure to thrips for six days, some plants were dusted with hydrophilic fumed silica particles (about 325 m²/g surface area), while other plants were sprayed with various chemical pesticides (lindane, 1 kg/1000 L; diazinon, 1 kg/1000 L; deltamethrin, 0.5 L/1000 L; endosulfan, 1 kg/1000 L; methomyl, 220 mL/1000 L; acephate, 850 L/1000 L; pyrazophos, 1.5 L/1000 L; bendiocarb, 1 kg/1000 L; malathion, 1.88 L/1000 L; and chlorpyrifos, 1 kg/1000 L). Some plants were not treated with any pesticide and served as controls. After twenty-four hours, the number of adult and larval thrips on each plant was counted. The relative numbers of larvae and adult thrips, as a percentage of control, on the plants with respect to each type of pesticide were determined. The results are summarized in FIG. 3.

These results demonstrate that fumed silica particles, applied as a post-infestation treatment, provided superior thrips control as compared to a range of pesticides with varying toxicity.

### EXAMPLE 5

This example illustrates the effectiveness of fumed silica particles, as compared to other silicates and clays, in reducing the ability of thrips to adhere to treated leaf surfaces.

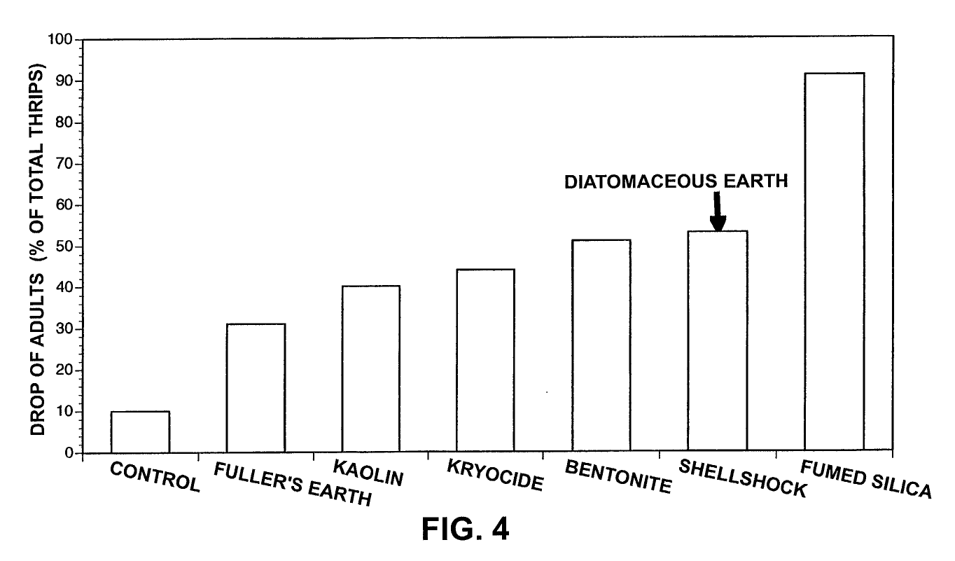

Hydrophilic fumed silica particles, silicates, or clays were applied as a dust to the upper surface of detached petunia leaves that were of the same age and approximate size. In turn, a leaf with each treatment received twenty adult thrips, which were allowed to move about on the leaf for 5 min to ensure contact with the dust before the leaf was inverted for 1 min over a sticky card, which could capture thrips that fall from the leaf. The number of thrips falling from leaves with respect to each treatment was determined and expressed as a percentage of the total number of thrips placed on the leaves. Tests with each treatment were repeated at least ten times in random order, and the average percentage of thrips falling from the leaves with respect to each treatment was calculated. The results are summarized in FIG. 4.

Ninety percent of the trips on leaves treated with hydrophilic fumed silica particles failed to adhere to the leaf surface when the leaf was inverted. In comparison, only 32-54% of thrips on leaves treated with the other silicates and clays failed to adhere to the leaf surface when the leaf was inverted. In particular, with respect to the most effective of the other silicates and clays tested, only about 50% of the thrips on leaves treated with the Shellshock® product, which is a diatomaceous earth that is coated with an adhesive in order to increase its ability to adhere to insects, failed to adhere to the leaf surface when the leaf was inverted.

### EXAMPLE 6

This example illustrates the effectiveness of fumed silica particles in protecting plants against viral transmission by WFT.

Petunia plants of the same age were uniformly trimmed to eight leaves of similar size. Half of the plants were dusted with hydrophilic fumed silica particles (about 325 m²/g surface area) and placed in rows, in alternate fashion with nontreated plants, on benches in a greenhouse. Each bench contained several flowering chrysanthemum and fava bean plants previously infested with thrips and thoroughly infected with the impatiens necrotic spot virus (INSV). After seven days of exposure, the number of viral lesions was counted on each of the treated and untreated petunia plants. This same test was repeated three times. The results are set forth in Table 3 as percentage reductions in viral lesions on the treated plants based on the viral lesions on the nontreated plants.

TABLE 3

| | % Reduction in | Level of |
|---|---|---|
| Trial | Virus Transmission | Significance (P) |
| 6A | 86.7 | <0.0004 |
| 6B | 92.5 | <0.0001 |
| 6C | 95.3 | 0.0046 |

These results indicate that hydrophilic fumed silica particles are highly effective in reducing virus transmission between plants by controlling thrips.

### EXAMPLE 7

This example illustrates the comparative effects on hydrophibic fumed silica particles, a hydrophobic silica gel, and chemical pesticides on thrips populations on plants and virus transmission under greenhouse conditions.

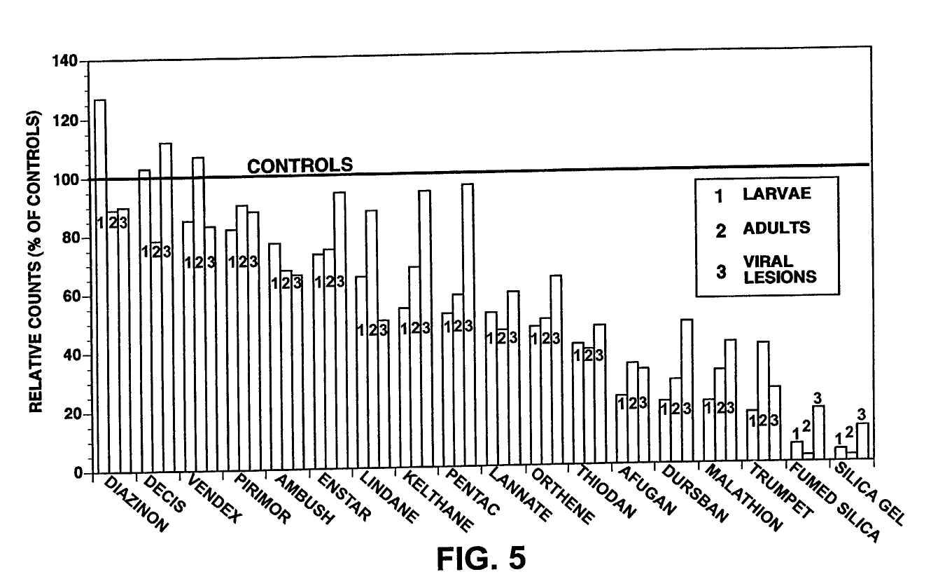

Hydrophilic fumed silica particles (surface area of about 325 m²/g) and a hydrophobic silica gel (surface area of about 300 m²/g) were dusted on uniformly trimmed petunia plants. Various chemical pesticides (diazinon, 1 kg/1000 L; deltamethrin, 0.5 L/1000 L; fenbutatin oxide, 1 kg/1000 L; permethrin, 200 mL/1000 L; pirimicarb, 0.5 kg/1000 L; kinoprene-S, 400 mL/1000 L; lindane, 1 kg/1000 L; dicofol, 1.25 L/1000 L; dienochlor, 650 mL/1000 L; methomyl, 220 mL/1000 L; acephate, 850 L/1000 L; endosulfan, 1 kg/1000 L; pyrazophos, 1.5 L/1000 L; chlorpyrifos, 1 kg/1000 L; malathion, 1.88 L/1000 L; and bendiocarb, 1 kg/1000 L) were sprayed on similarly trimmed petunia plants of the same age. Some such petunia plants were left untreated as controls. The treated and untreated plants were arranged alternately in rows on benches in a thrips-infested greenhouse containing virus-infected (impatiens necrotic spot virus) flowering chrysanthemum and fava bean plants. After seven days, the number of adult and larval thrips and the number of viral lesions were counted on each plant. For each treatment, the numbers of thrips (adult and larval) and viral lesions on the treated plants were divided by the numbers of thrips (adult and larval) and viral lesions, respectively, on the untreated plants to obtain percentage values. These results are plotted in FIG. 5.

The results demonstrate the superior efficacy of fumed silica particles regarding the control of thrips and virus transmission. The hydrophobic silica gel also significantly controlled the thrips and virus transmission, but it is not food-grade since it contains ammonium fluosilicate.

### EXAMPLE 8

This example illustrates the effect on efficacy towards thrips by fumed silica particles applied to plants and exposed to natural weathering conditions in a greenhouse for varying periods of time before exposure to thrips.

Uniformly aged and trimmed petunia plants were either dusted with hydrophilic fumed silica particles (about 325 m²/g surface area) or left untreated as controls. The treated and untreated plants were held in a thrips-free greenhouse compartment for 1, 2, or 3 weeks to allow for natural weathering of the hydrophilic fumed silica particles on the treated plants. After each weathering period, the treated plants and the untreated control plants were alternated in rows on benches in a greenhouse containing thrips-infested flowering chrysanthemum plants. After seven days, the number of larval thrips was counted on each plant. The results with the treated plants are set forth in Table 4 as percent reductions in larval thrips based on larval thrips on untreated control plants.

TABLE 4

| | Weeks of | % Reduction | Level of |
|---|---|---|---|
| Trial | Weathering | in Larvae | Significance (P) |
| 8A | 1 | 99.5 | <0.0001 |
| 8B | 2 | 93.8 | <0.0001 |
| 8C | 3 | 99 | <0.0001 |

These results demonstrate that the fumed silica particles remain highly effective in thrips control even after a few weeks, and likely longer, of on-plant weathering under greenhouse conditions.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

what is claimed is:

1. A method of protecting a plant from thrips comprising the step of applying silica particles to a plant or the plant's surrounding area, wherein the silica particles are fumed silica particles or silica aerogel particles to thereby protect the plant from thrips.

2. The method of claim 1, wherein the silica particles are fumed silica particles.

3. The method of claim 2, wherein the fumed silica particles have a surface area of about 50 m²/g to about 420 m²/g.

4. The method of claim 3, wherein the fumed silica particles have a surface area of about 300 m²/g to about 350 m²/g.

5. The method of claim 1, wherein the silica particles are silica aerogel particles.

6. The method of claim 5, wherein the silica aerogel particles have a surface area of about 200 m²/g to about 1100 m²/g.

7. The method of claim 1, wherein the plant is infested with thrips, and the method eradicates thrips from a plant.

8. The method of claim 1, wherein the method inhibits feeding on the plant by thrips.

9. The method of claim 1, wherein the method inhibits egg laying by thrips.

10. The method of claim 1, wherein the method prevents virus transmission by thrips.

11. The method of claim 1, wherein the thrips comprise adults and larvae.

12. The method of claim 1, wherein the plant is an ornamental plant.

13. The method of claim 12, wherein the plant is an ornamental plant selected from the group consisting of African violet, alstreomeria, aster, azalea, begonia, cacti, calceolaria, celosia, cineraria, cyclamen, chrysanthemum, dalia, exacum, gladiolus, geranium, gerbera, gloxinia, gypsophila, hibiscus, hydrangea, impatiens, kalanchoe, lily, lisianthus, oxalis, primula, petunia, poinsettia, rose, snapdragon, stocks, and stephanotis.

14. The method of claim 1, wherein the plant is a vegetable or fruit plant.

15. The method of claim 14, wherein the plant is a vegetable or fruit plant selected from the group consisting of citrus, pear, tomato, bean, soybean, cotton, tobacco, onion, cucumber, peanut, cabbage, cauliflower, broccoli, herb, lettuce, and pepper.

16. The method of claim 1, wherein the silica particles are applied to the leaves of the plant.

17. The method of claim 16, wherein the silica particles are applied to the upper surface of the leaves.

18. The method of claim 16, wherein the silica particles are applied to the plant's surrounding area.

19. The method of claim 1, wherein the silica particles are applied to the plant's surrounding area.

20. The method of claim 1, wherein the silica particles have an average aggregate particle size of about 300 nm or less.

21. The method of claim 1, wherein the silica particles are in a dry form.

22. The method of claim 1, wherein the silica particles are in the form of a liquid dispersion.

23. The method of claim 22, wherein the liquid dispersion comprises about 0.5 g to about 40 g of silica particles per liter of a liquid carrier.

24. The method of claim 23, wherein the liquid carrier is water.

25. The method of claim 24, wherein the liquid dispersion further comprises a dispersant or wetting agent.

26. The method of claim 1, wherein the method further comprises the step of removing the silica particles with water from the plant or the plant's surrounding area.

27. The method of claim 1, wherein the silica particles are applied to the plant in combination with an effective amount of another agent that protects the plant from thrips.
